(19) United States
(12) Patent Application Publication
(10) Pub. No.: US 2024/0390261 A1
(43) Pub. Date: Nov. 28, 2024

(54) HAIR CARE COMPOSITIONS, METHOD FOR PROVIDING IMPROVED CURLS DEFINITION TO THE HAIR AND METHOD OF TREATING HAIR

(71) Applicant: L'OREAL, Paris (FR)

(72) Inventors: Cyrus ANDRIOLO, Rio de Janeiro (BR); Celso ABREU, Rio de Janeiro (BR); Fernando GUIMARAES, Rio de Janeiro (BR); Carlos GRANADOS, Chevilly-Larue (FR); Erika PETALI, Rio de Janeiro (BR)

(21) Appl. No.: 18/696,604

(22) PCT Filed: Sep. 30, 2021
(86) PCT No.: PCT/BR2021/050422

§ 371 Date: Mar. 28, 2024

(51) Int. Cl. A61K 8/9789; A61K 8/34; A61K 8/365; A61K 8/368; A61K 8/44; A61K 8/55; A61K 8/92; A61Q 5/06

(52) U.S. Cl. CPC A61K 8/9789; A61K 8/345; A61K 8/365; A61K 8/368; A61K 8/44; A61K 8/55; A61K 8/922; A61Q 5/06

(57) ABSTRACT

The present disclosure is directed to hair care compositions, including at least one organic acid, at least one compound with at least one hydroxyl-group, at least one amino acid, at least one vegetal resin, and water. The present disclosure is also related to a method of treating hair, particularly curly hair.

### FIELD OF THE DISCLOSURE

The present disclosure is directed to hair care compositions comprising at least one organic acid, at least one compound with at least one hydroxyl-group, at least one amino acid, at least one vegetal resin, and water. The present disclosure is also related to a method of treating hair, particularly curly hair.

### BACKGROUND

People have been using several cosmetic and personal care compositions to enhance the look and feel of keratin fibers, such as the hair.

Particularly regarding curly hair, consumers are always dealing with problems related to the definition of the curls such as frizz, volume and shape. Curly hair tends to become frizzy and significantly expand in volume when exposed to high humidity conditions, chemical treatments, among others. Additionally, the shape and definition of the curls are lost after sleeping.

Many attempts to developed products to discipline, define and control the hair, in particular curly hair, enhancing/maintaining curl definition, curl retention, and curl regularity, without leaving the hair feeling and/or looking unnatural and stiff were made, but such products resulted in additional problems or maintenance difficulty.

For example, the products of the state of the art usually require high amount of product to give good combing and hair feel. But, considering that the effect of such products is not long lasting, the consumers have to reapply the product several times during the day or after sleeping in order to regain definition and frizz control, resulting in the build-up of the product on the hair's surface, resulting in a heavy aspect, wet look, unnatural touch and greasy sensation, since such products do not penetrate the keratin fibers and remain on the surface of the hair fiber.

In this sense, there is a need in the market for curly hair products that define the curls in a lasting way, without the necessity of reapplying the product until the next wash or after sleeping, also decreasing the need to use large amounts of the product between the washes.

Thus, based on the market's needs, the inventors of the present disclosure surprisingly and unexpectedly achieved hair care compositions comprising (a) at least one organic acid, (b) at least one compound with at least one hydroxyl-group, (c) at least one amino acid, (d) at least one vegetal resin, and (e) water, that are able to penetrate the hair fiber without building-up on the fiber's surface, while also providing curl definition until the next wash, long lasting of definition of the curls, frizz control, natural look and desirable volume, and that does not look heavy on the hair.

### SUMMARY

The present disclosure relates to hair care compositions comprising (a) at least one organic acid, (b) at least one compound with at least one hydroxyl-group, (c) at least one amino acid, (d) at least one vegetal resin, and (e) water, that are able to penetrate the hair fiber without building-up on the fiber's surface, while also providing curl definition until the next wash, long lastingness of the definition of the curls, frizz control, natural look and desirable volume, and that does not look or feel heavy on the hair.

Also, the present disclosure relates to a method for providing improved curls definition to the hair.

### BRIEF DESCRIPTION OF THE DRAWINGS

FIG. 1 relates to the results of Comparative Test 1.

FIG. 2 demonstrates the results of Comparative Test 2.

FIG. 3 demonstrates the results of the pH Analysis Test.

### DETAILED DESCRIPTION

The present disclosure is directed to a hair care composition, mainly for curly hair, comprising:
(a) at least one organic acid;
(b) at least one compound with at least one hydroxyl-group;
(c) at least one amino acid;
(d) at least one vegetal resin; and
(e) water.

In an embodiment, the at least one organic acid is selected from short chain organic acids, preferably α-hydroxy acids (AHAs), beta hydroxy acids, and a mixture thereof. In an embodiment, the at least one organic acid of the hair care composition of the present disclosure is selected from tartaric acid, lactic acid, glutaric acid, etidronic acid, salicylic acid, glycolic acid, and mixtures thereof.

Also, the amount of the at least one organic acid ranges from about 0.5% to about 8.0% by weight, preferably from about 1.0% to about 7.0% by weight, more preferably from about 2.0% to about 6.0% by weight, even more preferably from about 2.5% to about 4.0% by weight, and most preferably from about 3.0% to about 4.0% by weight, based on the total weight of the composition.

The hair care composition according to the present disclosure also comprises at least one compound with at least one hydroxyl-group. Without being limited to any one theory, it is believed that the at least one compound with at least one hydroxyl-group acts as a penetrating agent, that is, a compound that is capable of forming the dipole-dipole interaction with the keratin fibers, such as hair, and binding to polar sites on the surface of said keratin fiber and decrease the shielding, thus promoting penetration of the hair care composition into the keratin fibers.

In an embodiment, the at least one compound with at least one hydroxyl-group of the hair care composition of the present invent disclosure ion is selected from polyol, C₂ to C₆ monoalcohols, and a mixture thereof.

In an embodiment, the at least one compound with at least one hydroxyl-group of the hair care composition of the present disclosure is selected from polyols. In an embodiment, the polyol is a glycol selected from propylene glycol, dipropylene glycol, butylene glycol, pentylene glycol, hexylene glycol, glycerin and mixtures thereof. In an embodiment, the polyol comprises propylene glycol and dipropylene glycol. In an embodiment, the polyol is glycerin.

In an embodiment, the at least one compound with at least one hydroxyl-group of the hair care composition of the present disclosure is selected from C₂ to C₆ monoalcohols. In an embodiment, the C₂ to C₆ monoalcohols include ethanol and/or propanol and/or butanol and/or hexanol.

In an embodiment, the at least one compound with at least one hydroxyl-group of the hair care composition of the present disclosure comprises a mixture of polyols and C₂ to C₆ monoalcohols. In an embodiment, the at least one compound with at least one hydroxyl-group of the hair care composition of the present disclosure comprises propylene glycol and ethanol. In an embodiment, the at least one compound with at least one hydroxyl-group of the hair care composition of the present disclosure comprises dipropylene glycol and ethanol. In an embodiment, the at least one compound with at least one hydroxyl-group of the hair care composition of the present disclosure comprises glycerin and ethanol.

The amount of the at least one compound with at least one hydroxyl-group ranges from about 0.1% to about 20.0% by weight, preferably from about 0.3% to about 15.0% by weight, or from about 0.5% to about 12.0% by weight, or from about 0.5% to about 11.0% by weight, or from about 1.0% to about 11.0% by weight, based on the total weight of the composition.

According to the present disclosure, the at least one amino acid is selected from arginine.

The amount of the at least one amino acid ranges from about 0.1% to about 7.0% by weight, preferably from about 0.1% to about 6.0% by weight, more preferably from about 0.1% to about 5.0% by weight, even more preferably from about 0.5% to about 3.0% weight, based on the total weight of the composition. Other suitable ranges are from about 0.5% to about 2.0% by weight, or from about 0.7% to about 1.2% by weight, based on the total weight of the composition.

In an embodiment, the at least one vegetal resin is selected from Copaiba Officinalis (Balsam Copaiba) resin (oil resin or resin form).

The amount of the at least one vegetal resin ranges from about 0.1% to about 7.0% by weight, preferably from about 0.1% to about 5.0% by weight, preferably from about 0.15% to about 4.0% by weight, preferably from about 0.17% to about 4.0% by weight, or such as at about 0.1, about 0.17, about 0.2, about 0.3, about 0.36, about 0.4, about 0.5 to about 4.0% by weight, based on the total weight of the composition.

In a preferred embodiment, the hair care composition according to the present disclosure further comprises at least one vegetal oil, in an amount ranging from about 0.1% to about 7.0% by weight, preferably from about 0.1% to about 6.0% by weight, preferably from about 0.15% to about 5.0% by weight, preferably from about 0.15% to about 4.8% by weight, or such as at about 0.1, about 0.2, about 0.3, about 0.36, about 0.4, about 0.5 to about 4.8% by weight, based on the total weight of the composition.

### Organic Acid

The at least one organic acid of the compositions can be selected from alpha hydroxy acids, beta hydroxy acids, and a mixture thereof.

The term “alpha-hydroxy acid” is understood to mean, according to the present disclosure, a carboxylic acid having at least one hydroxyl functional group occupying an alpha-position on said acid (carbon adjacent to a carboxylic acid functional group). A beta-hydroxy acid is closely related to an alpha-hydroxy acid except the carboxylic and hydroxyl functional groups are separated by two carbon atoms. A hydroxy acid can be present in the final composition in the form of the free acid and/or in the form of one of its associated salts (salts with an organic base or an alkali metal, in particular), especially according to the final pH imposed on the composition.

The alpha hydroxy acid present in the composition, according to the disclosure, includes, but is not limited to, one or more of tartaric acid, glycolic acid, citric acid, lactic acid, methyllactic acid, glucuronic acid, pyruvic acid, 2-hydroxybutanoic acid, 2-hydroxypentanoic acid, 2-hydroxyhexanoic acid, 2-hydroxyheptanoic acid, 2-hydroxyoctanoic acid, 2-hydroxynonanoic acid, 2-hydroxydecanoic acid, 2-hydroxyundecanoic acid, 2-hydroxydodecanoic acid, 2-hydroxytetradecanoic acid, 2-hydroxyhexadecanoic acid, 2-hydroxyoctadecanoic acid, 2-hydroxytetracosanoic acid, 2-hydroxyeicosanoic acid, mandelic acid, phenyllactic acid, gluconic acid, galacturonic acid, aleuritic acid, ribonic acid, tartronic acid, malic acid, fumaric acid, their salts and their mixtures. It is also possible to use mixtures of these various acids.

In some embodiments, the composition includes alpha hydroxy acids comprising glycolic acid and one or more of citric acid and lactic acid. When combined, the more than one alpha hydroxy acids may be present in the same or in different amounts.

The beta hydroxy acid, when present in the composition according to the disclosure, includes, but is not limited to, one or more of salicylic acid and derivatives thereof (including 5-n-octanoylsalicylic acid, salicylate, sodium salicylate, and willow extract), beta hydroxybutanoic acid, tropic acid, and trethocanic acid.

In some embodiments, the composition includes tartaric acid.

### Compound with at Least One Hydroxyl-Group

The compound with at least one hydroxyl-group can be selected from polyols, and/or C₂ to C₆ monoalcohols such as ethyl alcohol, isopropyl alcohol, propyl alcohol, ethylene glycol, propylene glycol, butylene glycol, hexylene glycol, propane diol, and glycerin.

Further non-limiting examples include alkanediols (polyhydric alcohols) such as glycerin, 1,2,6-hexanetriol, trimethylolpropane, ethylene glycol, propylene glycol, diethylene glycol, triethylene glycol, tetraethylene glycol, pentaethylene glycol, dipropylene glycol, 2-butene-1,4-diol, 2-ethyl-1,3-hexanediol, 2-methyl-2,4-pentanediol, caprylyl glycol, 1,2-hexanediol, 1,2-pentanediol, and 4-methyl-1,2-pentanediol; alkyl alcohols having 1 to 4 carbon atoms such as ethanol, methanol, butanol, propanol, isopropanol, and a mixture thereof.

In some cases, the compound with at least one hydroxyl-group may be selected from the group consisting of one or more glycols, C1-4 alcohols, glycerin, and a mixture thereof. In some cases, the compound with at least one hydroxyl-group is selected from the group consisting of hexylene glycol, propylene glycol, dipropylene glycol, caprylyl glycol, glycerin, isopropyl alcohol, and a mixture thereof.

Polyhydric alcohols are useful. Examples of polyhydric alcohols include glycerin, ethylene glycol, diethylene glycol, triethylene glycol, propylene glycol, dipropylene glycol, tripropylene glycol, 1,3-butanediol, 2,3-butanediol, 1,4-butanediol, 3-methyl-1,3-butanediol, 1,5-pentanediol, tetraethylene glycol, 1,6-hexanediol, 2-methyl-2,4-pentanediol, polyethylene glycol, 1,2,4-butanetriol, 1,2,6-hexanetriol, and a mixture thereof.

Polyol compounds may also be used. Non-limiting examples include the aliphatic diols, such as 2-ethyl-2-methyl-1,3-propanediol, 3,3-dimethyl-1,2-butanediol, 2,2-diethyl-1,3-propanediol, 2-methyl-2-propyl-1,3-propanediol, 2,4-dimethyl-2,4-pentanediol, 2,5-dimethyl-2,5-hexanediol, 5-hexene-1,2-diol, and 2-ethyl-1,3-hexanediol, and a mixture thereof.

### Amino Acid

The compositions according to the disclosure comprise at least one amino acid. As used herein, the term “amino acid” includes amino acids such as proteinogenic amino acids, amino sulfonic acids, and salts thereof.

Amino acids are simple organic compounds containing both a carboxylic acid group (—COOH) and an amino group (—NH₂). Amino sulfonic acids are simple organic compounds containing both a sulfonic acid group (—SO₂OH) and an amino group (—NH₂). Accordingly, the amino acids useful according to the disclosure may, in certain embodiments, be selected from compounds of Formula (I) and Formula (II):

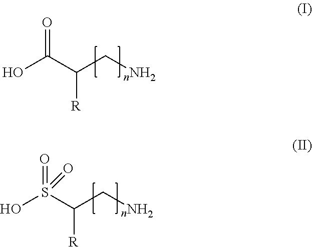

wherein:
R represents a hydrogen atom, a linear or branched, preferably linear, C₁-C₅ alkyl group, said alkyl group being optionally substituted with at least one group chosen from hydroxyl, —C(O)—OH, —S(O)₂—OH, —C(O)—O— and M⁺, and S(O)₂—O⁻ and M⁺, with M⁺ representing a cationic counter-ion such as an alkali metal, alkaline earth metal, or ammonium, and
n is 0 or 1.

The amino acids may be in their non-ionized form (I) and (II) or in their ionized or betaine form (l′) and (II′):

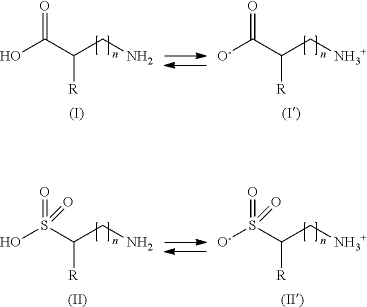

wherein R and n are as defined above.

The one or more amino acids may also be in their conjugate base form (Ib) and (IIb):

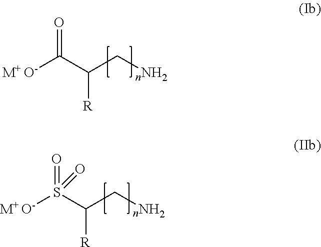

wherein R and n are as defined above.

Well-known amino acids include the twenty amino acids that form the proteins of living organisms (standard proteinogenic amino acids): alanine, arginine, asparagine, aspartic acid, cysteine, glutamic acid, glutamine, glycine, histidine, isoleucine, leucine, lysine, methionine, phenylalanine, proline, serine, threonine, tryptophan, tyrosine, and valine. The amino acids of the instant disclosure, however, are not limited to the standard proteinogenic amino acids.

Non-limiting examples of amino sulfonic acids include aminomethane sulfonic acid, 2-aminoethane sulfonic acid (taurine), aminopropane sulfonic acid, aminobutane sulfonic acid, aminohexane sulfonic acid, aminoisopropyl sulfonic acid, aminododecyl sulfonic acid, aminobenzene sulfonic acid, aminotoulene sulfonic acid, sulfanilic acid, chlorosulfanilic acid, diamino benzene sulfonic acid, amino phenol sulfonic acid, amino propyl benzene sulfonic acid, amino hexyl benzene sulfonic acid, and a mixture thereof.

In some cases, charged amino acids may be used. Non-limiting examples of charged amino acids include arginine, lysine, aspartic acid, and glutamic acid. In some cases, polar amino acids are useful. Non-limiting examples of polar amino acids include glutamine, asparagine, histidine, serine, threonine, tyrosine, cysteine, methionine, and tryptophan.

In some cases, hydrophobic amino acids may be employed. Non-limiting examples of hydrophobic amino acids include alanine, isoleucine, leucine, phenylalanine, valine, proline, and glycine.

### Vegetal Resin

Preferably, the at least one vegetal-based ingredient of the present disclosure is Copaifera officinalis (BALSAM COPAIBA) RESIN (INCI name) which can be in the form of a Copaiba oil resin or Copaiba resin. Copaifera officinalis (BALSAM COPAIBA) RESIN is commercially available under the tradename of RAIN FOREST RF3310 (also known by the chemical name of Copaiba Oleoresin) or under the tradename of RAIN FOREST 03370 (also known by the chemical name of Copaiba resin), both from the supplier Beraca Ingredientes Naturais.

### Vegetal Oil

In a non-limitative and preferred embodiment, the hair care composition of the present disclosure further comprises at least one vegetal oil, which is selected from coconut oil, almond oil, aloe vera oil, avocado oil, camelina oil, coffee oil, castor oil, grapeseed oil, jojoba oil, basil oil, bay oil, bergamot oil, black pepper essential oil, birch oil, camphor, cannabis oil, caraway oil, cardamom seed oil, carrot seed oil, chamomile oil, calamus root oil, cinnamon oil, citronella oil, clary sage, clove leaf oil, coffee, coriander, costmary oil, cranberry seed oil, cubeb, cumin oil, dill oil, eucalyptus oil, fennel seed oil, fenugreek oil, fir, frankincense oil, geranium oil, ginger oil, grapefruit oil, grapeseed oil, henna oil, horseradish oil, jasmine oil, juniper berry oil, lavender oil, lemon oil, lemongrass, marjoram, melaleuca, lemon balm oil, mountain savory, mugwort oil, mustard oil, myrrh oil, myrtle, neem tree oil, neroli, nutmeg, orange oil, oregano oil, orris oil, palo santo, parsley oil, peppermint oil, pine oil, plum oil, rose oil, rosehip oil, rosemary oil, rosewood oil, sandalwood oil, spruce, star anise oil, tarragon oil, tea tree oil, thyme oil, and mixtures thereof.

In some embodiments, the suitable vegetal oils for use in the hair care compositions of the present disclosure are selected from coconut oil, soybean oil, canola oil, rapeseed oil, corn oil, cottonseed oil, olive oil, palm oil, peanut oil, safflower oil, sesame oil, sunflower oil, linseed oil, palm kernel oil, wheatgerm oil, apricot kernel oil, pistachio oil, poppy oil, hazel nut oil, colza oil, cade oil, peach kernel oil, jojoba oil, Helianthus Annuus (sunflower) seed oil, and mixtures thereof.

When present, the vegetal oil can improve the penetration effect.

In a preferred embodiment, the at least one vegetal-based ingredient of the present disclosure is coconut oil, that is mainly composed by Lauric acid, Myristic Acid, Palmitic Acid, Oleic Acid, Caprylic Acid, Capric Acid. Other oils with the same composition could apply on the disclosure, such as Babassu oil and Murumuru butter e.

In a preferred embodiment, the hair care compositions of the present disclosure comprise a mixture of vegetal oils and vegetal resins, such as coconut oil and Copaiba Officinalis (Balsam Copaiba resin) (copaiba resin or copaiba oleoresin).

The pH of the hair care composition of the present disclosure is from about 1 to about 10, preferably from about 2 to about 7, or from about 2 to about 6. To achieve lasting of definition, the pH is about 3 to about 5, preferably from about 2.5 to about 4.5, or from about 3.5 to about 4.5, or at about 2, about, 3, about 3.3, about 3.5, about 4, about 4.5.

In a preferred embodiment, the composition of the disclosure is used as a leave-on or a rinse-off product to control frizz, improve curl definition, curl regularity and discipline, among other attributes.

In a preferred embodiment, the hair care composition of the present disclosure comprises:
(a) from about 0.5% to about 8.0% by weight of at least one organic acid selected from tartaric acid, glutaric acid, etidronic acid, salicylic acid, and a mixture thereof;
(b) from about 0.1% to about 20.0% by weight of at least one compound with at least one hydroxyl-group selected from polyol, C₂ to Ce monoalcohols, and a mixture thereof;
(c) from about 0.1% to about 7.0% by weight of arginine;
(d) from about 0.1% to about 7.0% by weight of at least one vegetal resin; water. (e) and

Another preferred embodiment of the hair care composition of the present disclosure on comprises:
(a) from about 0.5% to about 8.0% by weight of at least one organic acid selected from tartaric acid, glutaric acid, etidronic acid, salicylic acid, and a mixture thereof;
(b) from about 0.1% to about 20.0% by weight of at least one compound with at least one hydroxyl-group selected from propylene glycol, C₂ to C₆ monoalcohols, and a mixture thereof;
(c) from about 0.1% to about 7.0% by weight of arginine;
(d) from about 0.1% to about 7.0% by weight of at least one vegetal resin selected from Copaiba Officinalis (Balsam Copaiba) resin;
(e) water, and
(f) from about 1.0% to about 5.0% by weight of at least one vegetal oil selected from coconut oil.

Moreover, the present disclosure is directed to a method for providing improved curls definition to the hair, wherein the method comprises contacting the hair with the hair care cosmetic composition of the disclosure.

The hair care composition of the present disclosure can be manufactured from conventional cosmetic processes. An illustrative process comprises the following steps:
Step (1): in a vessel, adding from 50% to 70% of the water and the at least one organic acid, and mixing for about 10 minutes at room temperature;
Step (2): adding the at least one amino acid, mixing the ingredients until completely solubilization;
Step (4): adding the at least one compound with at least one hydroxyl-group, at least one vegetal resin, and optional vegetal oil, heating until about 65° C. to about 75° C. for about 15 minutes.
Step (5): adding the remaining water into the mixture, and cooling down.
Step (6): adding additional ingredients (if present) cooling down until about 25° C., adjusting the pH (if necessary) with a pH adjuster.

### Terms

As used herein, the expression “at least one” means one or more and thus includes individual components as well as mixtures/combinations.

Other than in the operating examples, or where otherwise indicated, all numbers expressing quantities of ingredients and/or reaction conditions are to be understood as being modified in all instances by the term “about,” meaning within +/−5% of the indicated number.

As used herein, all ranges provided are meant to include every specific range within, and combination of sub ranges between, the given ranges. Thus, a range from 1-5, includes specifically 1, 2, 3, 4 and 5, as well as sub ranges such as 2-5, 3-5, 2-3, 2-4, 1-4, etc. All ranges and values disclosed herein are inclusive and combinable. For examples, any value or point described herein that falls within a range described herein can serve as a minimum or maximum value to derive a sub-range, etc.

### Water

The hair care cosmetic compositions according to the present disclosure comprise water. The water used may be sterile demineralized water and/or deionized water and/or a floral water such as rose water, cornflower water, chamomile water or lime water, and/or a natural thermal or mineral water such as, for example: water from Vittel, water from the Vichy basin, water from Uriage, water from La Roche Posay, water from La Bourboule, water from Enghien-les-bains, water from Saint Gervais-les-Bains, water from Neris-les-Bains, water from Allevar-les-Bains, water from Digne, water from Maizieres, water from Neyrac-les-Bains, water from Lons-le-saunier, water from EauxBonnes, water from Rochefort, water from Saint Christau, water from Les Fumades, water from Tercis-les-Bains or water from Avene. Water may also comprise reconstituted thermal water, that is to say a water comprising trace elements such as zinc, copper, magnesium, etc., reconstituting the characteristics of a thermal water.

The hair care cosmetic composition according to the present disclosure may comprise water in an amount ranging from about 50% to about 99% by weight, preferably from about 65% to about 95% by weight, and most preferably from about 70% to about 92% by weight, relative to the total weight of the hair cosmetic composition.

### Additional Ingredients

In addition to the essential components described hereinbefore, the hair care composition of the present disclosure may further comprise any usual cosmetically acceptable ingredient, which may be chosen especially from such as cosmetically acceptable ingredients selected from perfume/fragrance, preserving agents, vitamins, vegetal extracts, pH-adjusters, organic solvents, and mixtures thereof.

A person skilled in the art will take care to select the optional additional ingredients and/or the amount thereof such that the advantageous properties of the composition according to the disclosure are not, or are not substantially, adversely affected by the envisaged addition.

The additional ingredients may represent from about 1% to about 70% by weight, based on the total weight of the composition.

By way of non-limiting illustration, the disclosure will now be described with reference to the following examples.

### Examples 1 to 4

Suitable hair care compositions according to the present disclosure are as Examples 1 to 4, as follows:

| | EX. 1 | EX. 2 | EX. 3 | EX. 4 |
|---|---|---|---|---|
| INGREDIENT | (%) | (%) | (%) | (%) |
| propylene glycol | 0.74 | 2.26 | 1.06 | 8.33 |
| arginine | 2.0 | 2.0 | 2.0 | 2.0 |
| tartaric acid | 3.0 | 3.0 | 3.0 | 3.0 |
| Copaifera officinalis (balsam | 0.17 | 0.19 | 0.22 | 3.93 |
| copaiba) resin (Copaiba resin) | | | | |
| Cocos nucifera (coconut) oil | 4.32 | 4.77 | 3.53 | 0.36 |
| ethanol | 0.55 | 4.11 | 1.72 | 1.89 |
| water | QS | QS | QS | QS |

### Examples 5 to 8

Other suitable hair care compositions according to the present disclosure are as Examples 5 to 8, as follows:

| | EX. 5 | EX. 6 | EX. 7 | EX. 8 |
|---|---|---|---|---|
| INGREDIENT | (%) | (%) | (%) | (%) |
| propylene glycol | 0.74 | 2.26 | 1.06 | 8.33 |
| arginine | 2.0 | 2.0 | 2.0 | 2.0 |
| tartaric acid | 3.0 | 3.0 | 3.0 | 3.0 |
| Copaifera officinalis (balsam | 0.17 | 0.19 | 0.22 | 3.93 |
| copaiba) resin (Copaiba Oleoresin) | | | | |
| Cocos nucifera (coconut) oil | 4.32 | 4.77 | 3.53 | 0.36 |
| ethanol | 0.55 | 4.11 | 1.72 | 1.89 |
| water | QS | QS | QS | QS |

### Tests

### Comparative Test 1

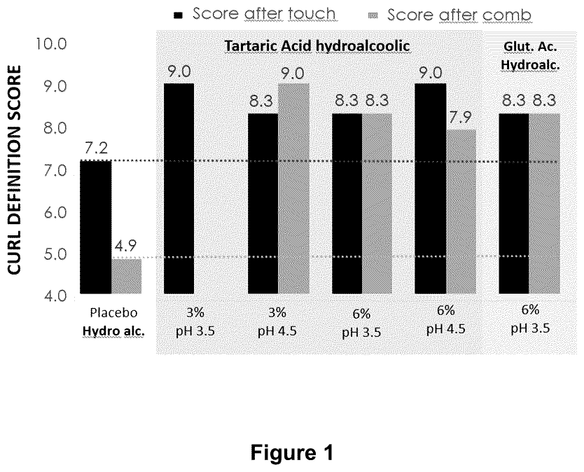

An in vitro comparative test was conducted to evaluate the curl definition on obtained with the hair care composition of Example 1 according to the present disclosure and a placebo composition comprising a hydroalcoholic medium without the organic acid (a) of the present disclosure. In FIG. 1, the black bars of the comparisons demonstrate the curl definition score after the application of the compositions containing 3.0% and 6.0% tartaric acid into the hair and after the touch (to simulate the normal touching of hair throughout the day), while the grey bars of the comparisons demonstrate the curl definition score after the application of the compositions into the hair and after combing. The curl definition score on swatches was obtained by image analysis.

As it can be seen, the composition according to the present disclosure with different pHs and different concentrations of tartaric acid resulted in better curl definition scores than the placebo composition, after touching and after combing.

### Comparative Test 2

Another comparative test was conducted in vitro to evaluate the curl definition after applying the hair care compositions according to Examples 1 to 4 of the present disclosure, in comparison with a hair treated only with a cleaning shampoo. The curl score definition on swatches was obtained by image analysis

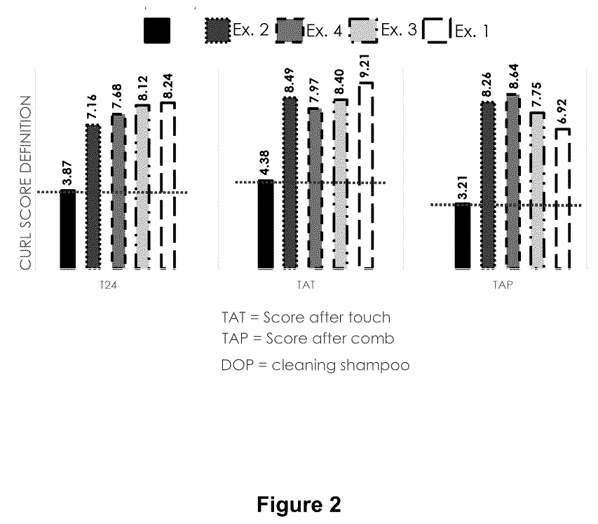

The results can be seen in FIG. 2. The horizontal dotted line represents the results after washing the hair with only a cleaning shampoo.

The results of the curl score definition after the application of the four compositions according to Examples 2 to 4 of the present disclosure were significantly superior when compared to the curl definition obtained after washing the hair with a cleaning shampoo.

### pH Analysis Test

In order to evaluate a possible impact of the pH of the hair care compositions on the curl definition, an in vitro test was conducted with compositions according to Example 1, varying the final pH. Six different pHs were analyzed: pH 2, pH 3.5, pH 4.5, pH 5, pH 6, and pH 7.

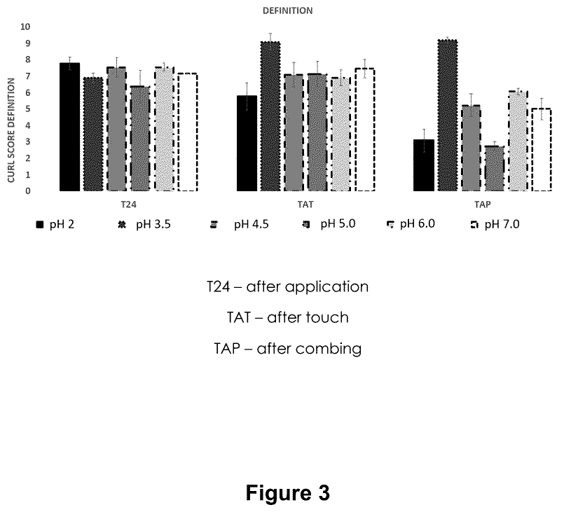

As can be seen from the results in FIG. 3, all hair care compositions according to Example 1 with different pH values presented good findings in curl definition after application and touch, being the most suitable results obtained with pH values of 3.5, 4.5, 6 and 7.

While the disclosure has been described with reference to a preferred embodiment, it will be understood by those skilled in the art that various changes may be made and equivalents may be substituted for elements thereof without departing from the scope of the disclosure. In addition, many modifications may be made to adapt a particular situation or material to the teachings of the disclosure without departing from the essential scope thereof. While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

1. A hair care composition, comprising:
(a) at least one organic acid;
(b) at least one compound with at least one hydroxyl-group;
(c) at least one amino acid;
(d) at least one vegetal resin; and
(e) water.

2. The composition, according to claim 1, wherein the organic acid is selected from short chain α-hydroxy acids (AHAs), beta hydroxy acids, and a mixture thereof.

3. The composition, according to claim 1, wherein the at least one organic acid is selected from tartaric acid, lactic acid, glutaric acid, etidronic acid, salicylic acid, glycolic acid, and mixtures thereof.

4. The composition, according to claim 1, wherein the amount of the at least one organic acid ranges from 0.5% to 8.0% by weight, based on the total weight of the composition.

5. The composition, according to claim 1, wherein the at least one compound with at least one hydroxyl-group is selected from polyols, C₂ to C₆ monoalcohols, and mixtures thereof.

6. The composition, according to claim 5, wherein the polyol is selected from propylene glycol, dipropylene glycol, butylene glycol, pentylene glycol, hexylene glycol, glycerin, and mixtures thereof.

7. The composition, according to claim 1, wherein the amount of the at least one compound with at least one hydroxyl-group ranges from 0.1% to 20% by weight, based on the total weight of the composition.

8. The composition, according to claim 1, wherein the at least one amino acid is arginine.

9. The composition, according to claim 1, wherein the amount of the at least one amino acid ranges from 0.1% to 7.0% by weight, preferably from 0.1% to 6.0% by weight, more preferably from 0.1% to 5.0% by weight, even more preferably from 0.5% to 3.0% weight, based on the total weight of the composition.

10. The composition, according to claim 1, wherein the amount of the at least one vegetal resin ranges from 0.1% to 7.0% by weight, preferably from 0.1% to 5% by weight, preferably from 0.15% to 4.0% by weight, preferably from 0.17% to 4.0% by weight, based on the total weight of the composition.

11. The composition, according to claim 1, wherein the vegetal resin includes Copaiba Officinalis (Balsam Copaiba) resin (oil resin or resin form).

12. The composition, according to claim 1, further comprising at least one vegetal oil.

13. The composition, according to claim 12, wherein the amount of the at least one vegetal oil ranges from 0.1% to 7.0% by weight, preferably from 0.1% to 6.0% by weight, preferably from 0.15% to 5.0% by weight, preferably from 0.15% to 4.8% by weight, based on the total weight of the composition.

14. The composition, according to claim 12, wherein the at least one vegetal oil includes coconut oil.

15. The composition, according to claim 1, wherein it further comprises cosmetically acceptable ingredients selected from perfume/fragrance, preserving agents, vitamins, vegetal extracts, pH-adjusters, organic solvents and mixtures thereof.

16. The composition, according to claim 1, wherein the pH of the composition ranges from 2 to 7.

17. A hair care composition, comprising:
(a) from 0.5% to 8.0% by weight of at least one organic acid selected from tartaric acid, lactic acid, glutaric acid, etidronic acid, salicylic acid, glycolic acid, and a mixture thereof;
(b) from 0.1% to 20.0% by weight of at least one compound with at least one hydroxyl-group selected from polyols, C₂ to C₆ monoalcohols, and a mixture thereof;
(c) from 0.1% to 7.0% by weight of arginine;
(d) from 0.1% to 7.0% by weight of at least one vegetal resin;
(e) water; and
(f) optionally, from 0.1% to 7.0% by weight of at least one vegetal oil, all weights based on the total weight of the composition.

18. A method for providing improved curls definition to the hair, wherein the method comprises contacting the hair with the hair care cosmetic composition, as defined in claim 1.

19. A method of treating hair, wherein the method comprises contacting the hair with the hair care cosmetic composition, as defined in claim 1.
